(19) United States
(12) Patent Application Publication
(10) Pub. No.: US 2015/0017687 A1
(43) Pub. Date: Jan. 15, 2015

(54) METHOD FOR OBTAINING A GLYCOPROTEIN COMPOSITION

(71) Applicant: DR. REDDY'S LABORATORIES LIMITED, Qutubullapur (IN)

(72) Inventors: Asma Ahmed, Secunderabad (IN); Rekha Hariharan, Mumbai (IN); Sama Naresh Babu, Ghatkesar (IN)

(21) Appl. No.: 14/373,853

(22) PCT Filed: Jan. 17, 2013
(86) PCT No.: PCT/IB2013/050444

§ 371 Date: Jul. 22, 2014

(30) Foreign Application Priority Data
Jan. 30, 2012 (IN) 333/CHE/2012

(51) Int. Cl. C07K 16/28

(52) U.S. Cl. CPC C07K 16/2896; C07K 2317/14
USPC 435/70.1

(57) ABSTRACT

Provided is a method for producing glycoprotein with a specific glycoform compostion. The desired glycoform profile is brought about Iby altering the culture conditions on the basis of IVCC rather than the age of the culture. Further, the method renders a high product yield.

### RELATED APPLICATION

This application is related to and takes priority from Indian Provisional Application 333/CHE/2012 filed 30 Jan. 2012 and is herein incorporated in its entirety.

### INTRODUCTION

The invention describes a method for obtaining a glycoprotein with a particular glycoform composition by altering culture conditions on the basis of integral of viable cell count (IVCC) rather than the age of the culture. More specifically, the invention describes a cell culture process wherein cells in the production phase are cultured till attainment of particular IVCC, after which, temperature is reduced to obtain high product yield.

Protein glycosylation is one of the most important post-translation modifications associated with eukaryotic proteins. The two major types of glycosylation in eukaryotic cells are N-linked glycosylation, in which glycans are attached to the asparagine of the recognition sequence Asn-X-Thr/Ser, where “X” is any amino acids except proline, and O-linked glycosylation in which glycans are attached to serine or threonine. N-linked glycans are of further two types—high mannose type consisting of two N-acetylglucosamines plus a large number of mannose residues (more than 4), and complex type that contain more than two N-acetylglucosamines plus any number of other types of sugars. In both N- and O-glycosylation, there is usually a range of glycan structures associated with each site (microheterogeneity). Macroheterogeneity results from the fact that not all N-glycan or O- glycan consensus sequences (Asn-X-Ser/Thr for N-glycan and serine or threonine for O-glycan present in the glycoproteins) may actually be glycosylated. This may be a consequence of the competitive action of diverse enzymes during biosynthesis and are key to understanding glycoprotein heterogeneity. (Mariño, K., (2010) Nature Chemical Biology 6, 713-723).

The process of N-linked glycosylation begins co-translationally in the Endoplasmic reticulum where a complex set of reactions result in the attachment of Glc₃NAc₂Man₉ (3 glucose, 2 N-acetylglucosamine and 9 mannose) to a carrier molecule called dolichol, that is then transferred to the appropriate point (Asn 297)on the polypeptide (Schwarz, F. and Aebi M., (2011) Current Opinion in Structural Biology, 21:576-582; Burda, P. & Aebi M., (1999) Biochimica et BiophysicaActa (BBA) General Subjects Volume 1426, Issue 2, Pages 239-257). The glycan complex so formed in the ER lumen is modified by action of enzymes in the Golgi apparatus. If the saccharide is relatively inaccessible, it is likely to stay in the original high-mannose form. If it is accessible, then many of the mannose residues may be cleaved off and the saccharide further modified, resulting in the complex type N-glycans structure. In the cis-Golgi, mannosidase-1 may act to hydrolyze/cleave high mannose glycan, while further on, fucosyltransferase FUT-8 fucosylates the glycan in the medial-Golgi (Hanrue Imai-Nishiya (2007), BMC Biotechnology, 7:84).

Thus the sugar composition as well as the structural configuration of a glycan structure depends on the protein being glycosylated, the cells/cell lines, the glycosylation machinery in the Endoplasmic Reticulum and the Golgi apparatus, the accessibility of the machinery enzymes to the glycan structure, the order of action of each enzyme and the stage at which the protein is released from the glycosylation machinery.

In addition to the “in vivo” factors listed above, “external factors” may also affect the glycan structure and composition of a protein. These include the conditions in which the cell line expressing the protein is cultured, such as the medium composition, the composition and timing of the feed, osmolality, pH, temperature etc. Pacis et al has shown that higher osmolality may result in increase in the number of Man5 residues on recombinant antibodies, with a simultaneous reduction in G₀F and G₁F glycoforms, resulting in its faster clearance from the body and thereby reducing its efficacy (Pacis E., Yu, M., Autsen, J., Bayer, R., Li F., (2011) Bitechnol Bioeng 108 (10) 2348-2358).

Studies by Kaufman et al and Yoon et al show a reduction in protein sialylation upon decrease in temperature (Kaufman, H., Mazur X., Fussenegger, M., Bailey, J. E., (1999) Biotechnol Bioeng. 63, 573-578; Trummer, E., Fauland, K., et.al. (2006) Biotechnol Bioeng. 94 1045-1052); Yoon S. K., Song, J. Y., Lee, G. M., (2003) Biotechnol Bioeng. 82: 289-298). Further, reducing temperature can increase overall protein production by prolonging cell viability, which should, in principle, improve glycosylation. (Moore A, Mercer J, Dutina G, Donahue C J, Bauer K D, Mather J P, Etcheverry T, Ryll T. (1997), Cytotechnology. 23:47-54).

Likewise Borys et al has shown that a deviation from optimum pH results in decrease in the expression rate as well as the extent of glycosylation of proteins (Borys M. C., Linzer, D. I. H., Papoutsakis (1993), BIO/technology 11 720-724). The culture pH of a hybridoma cell line has been shown to affect the resulting galactosylation and sialylation of the monoclonal antibody (Muthing J, Kemminer S E, Conradt H S, Sagi D, Nimtz M, Karst U, Peter-Katalinic J. (2003). BiotechnolBioeng 83:321-334).

The structure and composition of the glycan moieties of a glycoprotein can have a profound effect on the safety and efficacy of therapeutic proteins, including its immunogenicity, solubility and half life. For instance, the absence of fucose in the glycan structure of the Fc region of the antibodies has been associated with higher antibody dependent cell mediated cytotoxicity (ADCC) activity, and presence of higher mannose glycans has been associated with faster clearance of glycoprotein from serum (Werner, R. G., Kopp, K. and Schlueter, M. (2007), 96: 17-22. doi: 10.1111/j.1651-2227.2007.00199.x). Removal of terminal galactose residues from the chimeric mouse—human IgG1 antibody (alemtuzumab) was shown to reduce complement dependent cytotoxicity (CDC), without effecting FcγR-mediated functions (Boyd, P. N., Lines, A. C. & Patel, A. K. (1995), Mol. Immunol.32, 1311-1318). Similarly, the (G₁F-G₁F) glycoform of rituximab triggered a CDC response twice as large as that triggered by the (G₀F-G₀F) glycoform (http://www.nature.com/nrd/journal/v8/n3/full/nrd2804.html-accessed on 23 Dec. 2011).

Hence given the role of the glycan structure and composition in the activity and efficacy of a glycoprotein on the one hand, and the array of factors that affect the glycan composition on the other, methods that control the glycan composition of glycoproteins would be beneficial.

The present invention describes a process of obtaining an antibody composition comprising a particular glycoform distribution wherein the culture conditions are altered upon attainment of particular IVCC.

### SUMMARY

A method for producing a glycoprotein having particular glycoform composition is described. The invention describes a process wherein cells are cultured to a certain IVCC, subsequent to which temperature is lowered and feed is added to attain an antibody composition comprising a particular glycoform distribution.

### BRIEF DESCRIPTION OF THE DRAWINGS

FIG. 1 is an illustration of effect of IVCC based temperature shift and feed addition on cell viability as described in Examples 1-2

FIG. 2 is an illustration of effect of IVCC based temperature shift and feed addition on antibody titer as described in Examples 1-2

FIG. 3 is an illustration effect of IVCC based temperature shift and feed addition on major glycoforms as described in Examples 1-2

### DETAILED DESCRIPTION OF THE INVENTION

Definitions

As used herein, “IVCC” or “Integral viable cell concentration” refers to cell growth over time or integral of viable cells with respect to culture time that is used for calibration of specific protein production. The integral of viable cell concentration can be increased either by increasing the viable cell concentration or by lengthening the process time.

The “viable cell count” or “cell viability” is defined as number of live cells in the total cell population. For e.g. by 35-40% viability it is meant that 35-40 percent of the cells are viable in the culture conditions at that point of time.

The “seeding density” is defined as the number of cells that are placed into a bioreactor during cell passage or during production stage.

The term “osmolality” as used herein is defined as a measure of the osmoles of solute per kilogram of solvent (osmol/kg) and may include ionized or non-ionized molecules and may change during the cell culture process for e.g. by addition of feed, salts, additives or metabolites.

The term “temperature shift” as used herein refers to any change in temperature during the cell culture process. For the purpose of this invention, the initial temperature of the cell culture process is higher than the final temperature i.e. cells are subjected to a temperature downshift wherein cells are first cultured at a higher temperature for certain time period after which temperature is reduced, and cells are cultured at this lower temperature for a fixed period of time.

The term “glycan” refers to a monosaccharide or polysaccharide moiety.

The term “glycoprotein” refers to protein or polypeptide having at least one glycan moiety. Thus, any polypeptide attached to a saccharide moiety is termed as glycoprotein.

The term “glycoform” or “glycovariant” have been used interchangeably herein, and refers to various oligosaccharide entities or moieties linked in their entirety to the Asparagine 297 (as per Kabat numbering) of the human Fc region of the glycoprotein in question, co translationally or post translationally within a host cell. The glycan moieties that may be added during protein glycosylation include M3, M4, M5-8, M3NAG etc. Examples of such glycans and their structures are listed in Table 1. However, Table 1 may not be considered as limitations of this invention.

The “glycoform composition” or distribution as used herein pertains to the quantity or percentage of different glycoforms present in a glycoprotein.

As used herein, “high mannose glycovariant” consists of glycan moieties comprising two N-acetylglucosamines and more than 4 mannose residues i.e. M5, M6, M7, and M8.

The “complex glycovariant” as used herein consists of glycan moieties comprising any number of sugars.

“Afucosylated glycovariants or glycoforms” described here, consists of glycan moieties wherein fucose is not linked to the non reducing end of N-acetlyglucosamine (for e.g. M3NAG, G₀, G_{1A}, G_{1B}, G₂).

G₀ as used herein refers to protein glycan not containing galactose at the terminal end of the glycan chain.

G₀F as described here consists of glycan moieties wherein fucose is linked to the non reducing end of N-acetylglucosamine.

TABLE I
Representative table of various glycans

| Glycan Structure | Code |
|---|---|
| 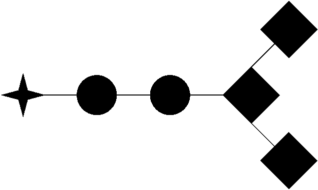 | M3 |
| 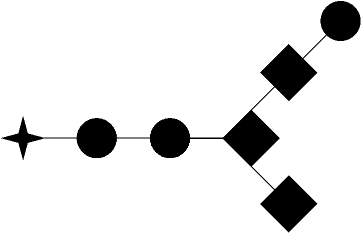 | M3NAG |
| 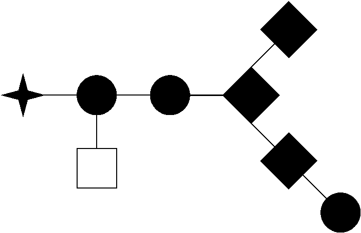 | M3NAGF |
| 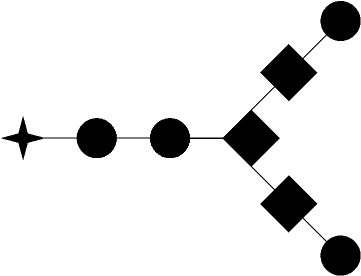 | G₀ |
| 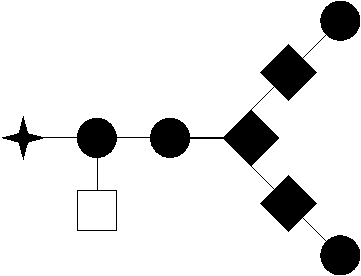 | G₀F |
| 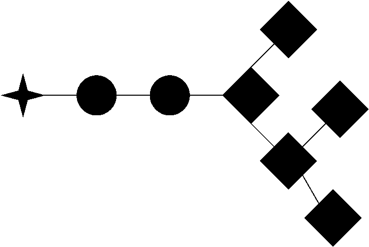 | M5 |
| 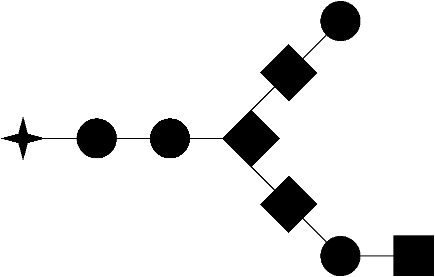 | G_{1A} |
| 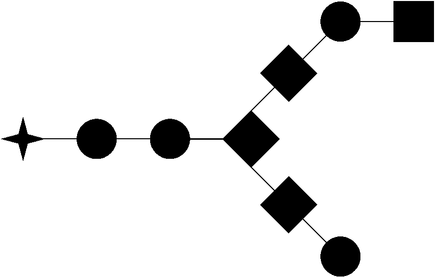 | G_{1B} |
| 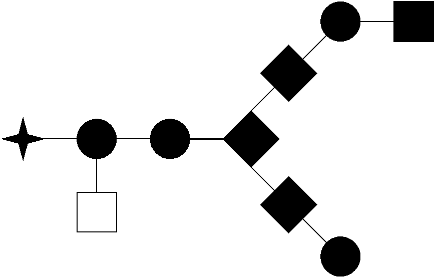 | G_{1A}F |
| 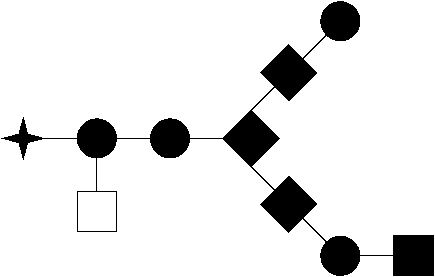 | G_{1B}F |
| 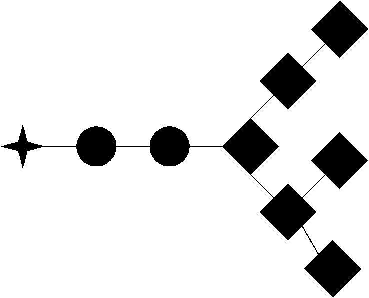 | M6 |
| 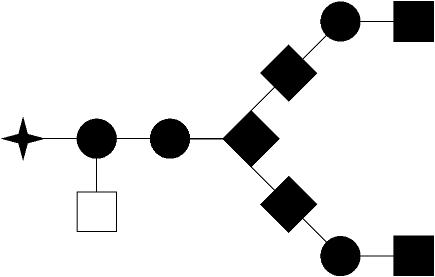 | G₂F |
| 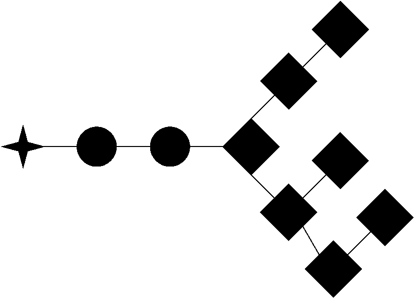 | M7 |
| 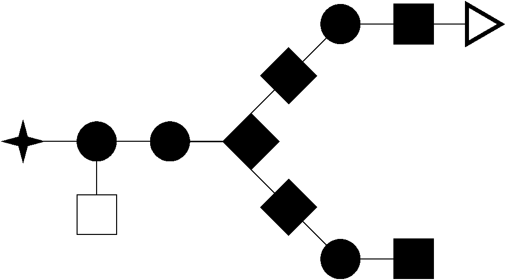 | G₂SF |
| 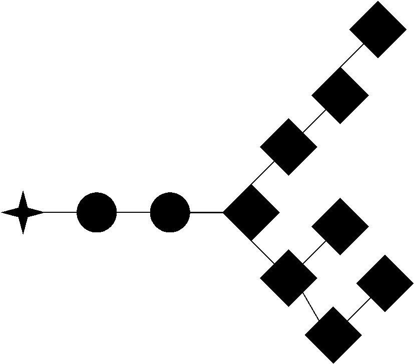 | M8 |
| 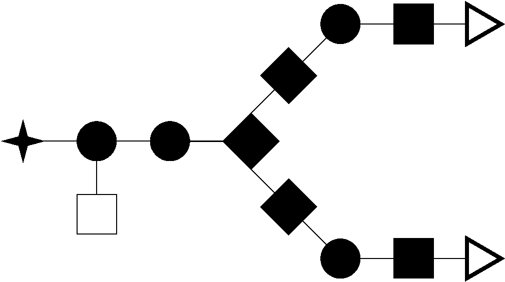 | G₂S₂F |

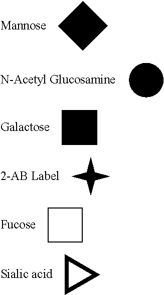

The present invention provides a method for obtaining a glycoprotein with a particular glycoform composition. In particular, the invention provides a cell culture process wherein cells are maintained at a particular temperature to attain a particular IVCC, after which, temperature is reduced to obtain a high product yield.

In one embodiment the present invention provides, a process for obtaining a glycoprotein composition comprising about 2.5% to about 3.9% high mannose glycans, about 1.8% to about 3.0% afucosylated glycans and about 45.7% to about 51.5% of G₀F glycan comprising culturing cells expressing said glycoprotein,
a) at a seeding density of about 0.65 to about 0.85 million cells/ml
b) at a first temperature, for a first period of time to obtain IVCC of about 4.0 to about 6.0
c) subjecting the cells to a second temperature, for second period of time and
d) recovering the protein from the cell culture

The shift in temperature may be accompanied by addition of nutrient feed, and the temperature is shifted towards lower values.

The cells may first be cultured at a temperature of about 35° C.-37° C. to obtain an IVCC of about 4.0 to about 6.0, followed by lowering of temperatures by about 2-7° C.

In particular the cells may be cultured at about 37° C. to obtain an IVCC of about 4.0 to 6.0, followed by shifting the temperature to about 35° C., accompanied by addition of feed.

In another embodiment, the present invention provides a process for obtaining a glycoprotein composition comprising about 5.2% to about 5.3% high mannose glycans, about 2.1% afucosylated glycans and about 47.4% to about 48.2% of G₀F glycan comprising, culturing cells expressing said glycoprotein,
a) at a seeding density of about 0.65 to about 0.85 million cells/ml
b) at a first temperature for a first period of time to attain IVCC of about 4.0 to about 6.0
c) subjecting cells to a second temperature for a second period of time to attain IVCC of 12- 14, followed by
d) addition of feed and culturing cells for a third period of time; and
e) recovering the protein from the cell culture.

The shift in temperature may be accompanied by addition of nutrient feed, and temperature is shifted towards lower values.

Various methods described in the art such as Wuhreret. al., Ruhaak L. R., and Geoffrey et. al. can be used for assessing glycovariants present in a glycoprotein composition (Wuhrer M. et al., Journal of Chromatography B, 2005, Vol. 825, Issue 2, pages 124-133; Ruhaak L. R., Anal BioanalChem, 2010, Vol. 397:3457-3481 and Geoffrey, R. G. et. al. Analytical Biochemistry 1996, Vol. 240, pages 210-226).

The feeds in the present invention may be added in a continuous, profile or a bolus manner. Also it may be that one or more feeds are in one manner (e.g. profile mode) and others are in second mode (e.g. bolus or continuous mode). Further, the feed may be composed of nutrients or other medium components that have been depleted of metabolized by the cells. It may include hormones, growth factors, ions vitamins, nucleoside, nucleotides, trace elements, amino acids, lipids or glucose. These supplementary components may be added at one time or in series of additions to replenish. Thus feed can be a solution of depleted nutrient(s), mixture of nutrient(s) or a mixture of cell culture medium/feed providing such nutrient(s).

In one aspect of the invention, concentrated cell culture media is used as a feed.

The cell culture media that are useful in the application include but are not limited to, the commercially available products PF CHO (HyClone®), PowerCHO® 2 (Lonza), Zap-CHO (Invitria), CD CHO, CDOptiCHO™ and CHO-S-SFMII (Invitrogen), ProCHO™ (Lonza), CDM4CHO™ (Hyclone), DMEM (Invitrogen), DMEM/F12 (Invitrogen), Ham's F10 (Sigma), Minimal Essential Media (Sigma), and RPMI-1640 (Sigma).

Certain aspects and embodiments of the invention are more fully defined by reference to the following examples. These examples should not, however, be construed as limiting the scope of the invention.

### EXAMPLE I

An anti-CD20 antibody was cloned and expressed in a CHO cell line as described in U.S. Pat. No. 7,381,560 which is incorporated herein by reference. The production bio-reactor is initiated with the rCHO cells at seeding density of 0.65-0.85 million cells/ml in POWER CHO2 (Lonza, Catalog no: 12-771Q)comprising 6 g/L galactose at 37° C., pH 7.05 at an osmolality of 350-390 mOSm/Kg. The cells are cultured to attain IVCC of 4-6 million cells/ml. Subsequently, temperature is lowered to 35° C. and simultaneously feed (4×POWER CHO2, 70 ml/L) is added. The cells are further cultured and harvested at 35-40% viability or at 288±12 hrs.

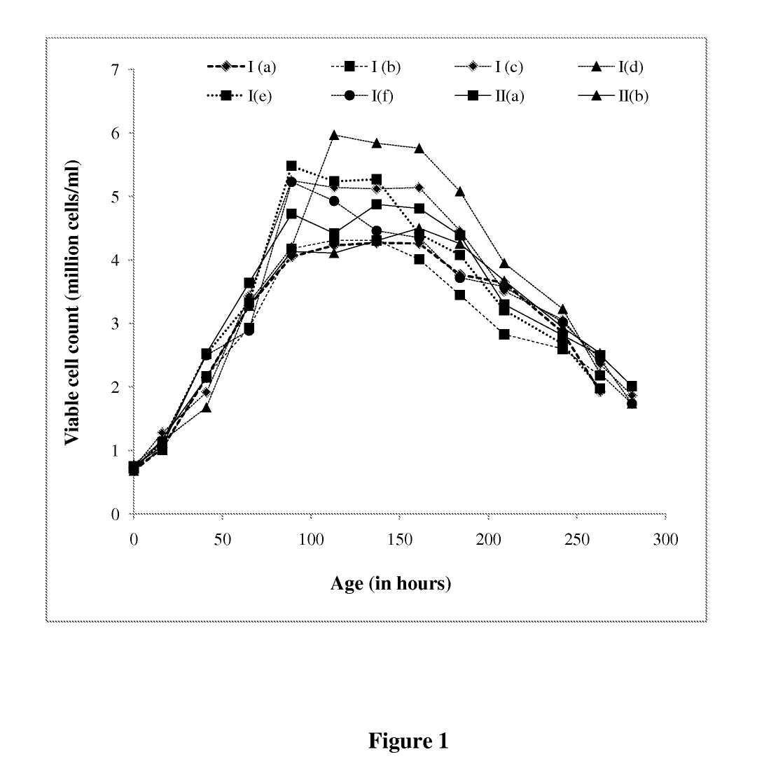

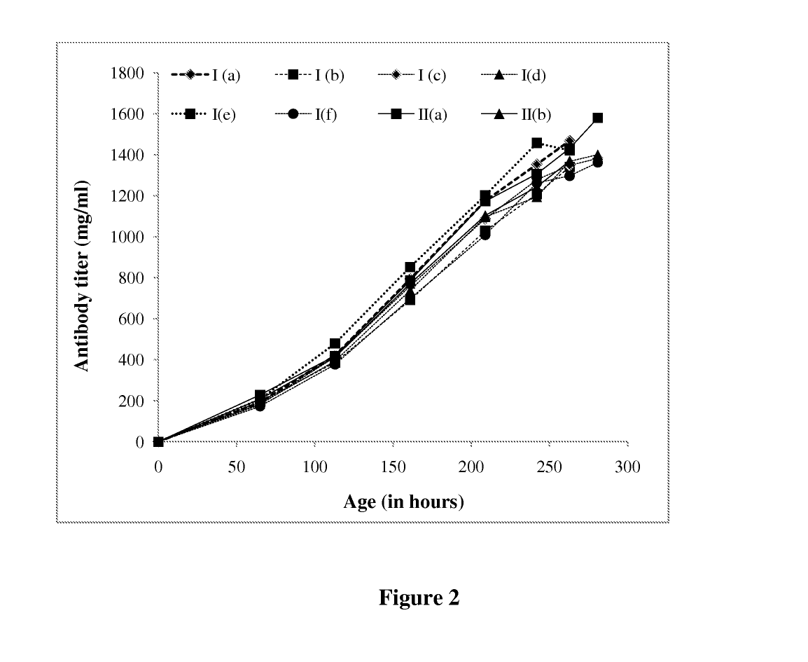

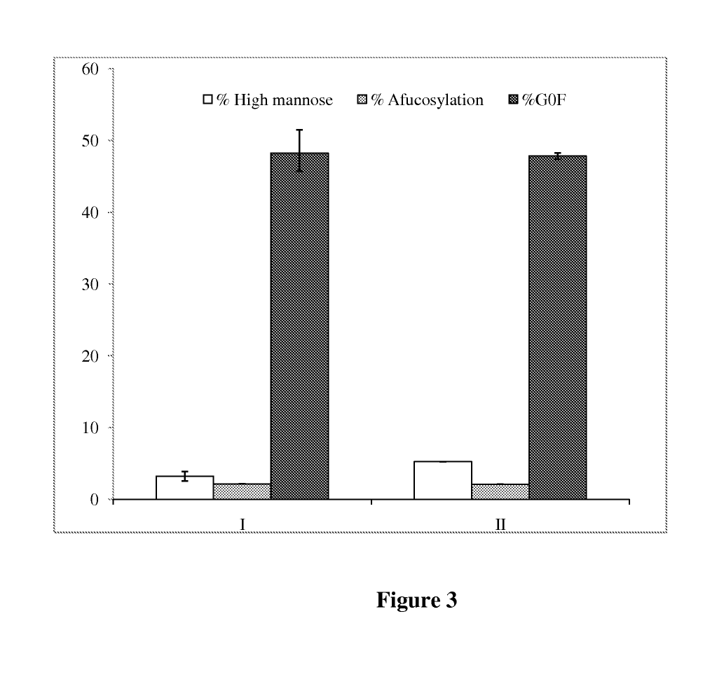

The above process was used to produce six independent batches of the anti-CD20 antibody (1a-1f). The results are shown in FIGS. 1-3. The average percentage viability, IVCC and antibody yield have been disclosed in Table II, and Table III shows the glycoform composition.

### EXAMPLE II

An anti-CD20 antibody was cloned and expressed in a CHO cell line as described in U.S. Pat. No. 7,381,560 which is incorporated herein by reference. The production bio-reactor is initiated with the rCHO cells at seeding density of 0.65-0.85 million cells/ml in POWER CHO2 (Lonza, Catalog no: 12-771Q) comprising 6 g/L galactose at 37° C., pH 7.05 at an osmolality of 350-390mOSm/Kg. The cells are cultured to attain IVCC of 4-6 million cells/ml. Subsequently, temperature is lowered to 35° C. and simultaneously feed (4×POWER CHO2, 70 ml/L) is added. The cells are further cultured to attain an IVCC of 12-14 million cells/ml. Subsequently, the second feed (4×POWER CHO2, 70 ml/L) is added. The cells are further cultured and harvested at 35-40% viability or at 288±12 hrs.

The process was used to produce two batches of the anti-CD20 antibody (IIa-IIb). The results are shown in FIGS. 1-3. The average percentage viability, IVCC and antibody yield have been disclosed in Table II, and Table III shows the glycoform composition.

TABLE II
% Viability of cells and Antibody concentration at the time of harvest.

| | % Viability of | | Final Antibody |
|---|---|---|---|
| | cells at harvest | IVCC | Concentration (mg/l) |
| Examples | Mean (range) | Mean (range) | Mean (range) |
| I (n = 6) | 42.2 (37.0-51.0) | 38.8 (33.0-44.0) | 1395.5 (1340.0-1468.0) |
| II (n = 2) | 48.0 (45.0-51.0) | 34.0 (35.0-33.0) | 1404.0 (1340.0-1468.0) |

TABLE III
Glycoform profile of antibodies

| | % High Mannose | % Afucosylation | |
|---|---|---|---|
| Examples | Mean (range) | Mean (range) | % G₀FMean (range) |
| I (n = 6) | 3.2 (2.5-3.9) | 2.16 (1.8-3.0) | 48.24 (45.7-51.5) |
| II (n = 2) | 5.25 | 2.12 | 47.8 (48.2-47.4) |

We claim:

1. A cell culture process for obtaining a glycoprotein composition comprising about 2.5% to about 3.9% high mannose glycans, about 1.8% to about 3.0% afucosylated glycans and about 45.7% to about 51.5% of G₀F glycans comprising culturing cells expressing said glycoprotein in a cell culture media
a) at a seeding density of about 0.65 to about 0.85 million cells/ml
b) at a first temperature for a first period of time to obtain an IVCC of about 4.0 to about 6.0
c) at a second temperature for a second period of time and
d) recovering glycoprotein from the cell culture.

2. A process according to claim 1, wherein cells are cultured in step b) at a temperature of about 35° C. to about 37° C.

3. A process according to claim 1, wherein cells are cultured in step b) at a temperature of about 37° C.

4. A process according to claim 1, wherein temperature in step c) is reduced in the range of about 2° C. to about 7° C.

5. A process according to claim 1, wherein cells are cultured in step c) at a temperature of about 35° C.

6. A process according to claim 1, wherein the process further comprises addition of a feed.

7. A process according to claim 1, wherein the cells are harvested at about 35% to about 40% cell viability or after about 276 to about 300 hours.

8. A cell culture process for obtaining a glycoprotein composition comprising about 5.2% to about 5.3% high mannose glycans, about 2.1% afucosylated glycans and about 47.4% to about 48.2% of G₀F glycan comprising culturing cells expressing said glycoprotein in a cell culture media
a) at a seeding density of about 0.65 to about 0.85 million cells/ml
b) at a first temperature for a first period of time to attain an IVCC of about 4.0 to about 6.0
c) at a second temperature for a second period of time to attain an IVCC of about 12.0 to about 14.0
d) addition of feed and culturing cells for a third period of time; and
e) recovering glycoprotein from the cell culture.

9. A process according to claim8, wherein cells are cultured in step b) at a temperature of about 35° C. to about 37° C.

10. A process according to claim 8, wherein cells are cultured in step b) at a temperature of about 37° C.

11. A process according to claim 8, wherein temperature in step c) is reduced in the range of about 2° C. to about 7° C.

12. A process according to claim8, wherein cells are cultured in step c) at a temperature of about 35° C.

13. A process according to claim 8, wherein the process further comprises addition of a feed.

14. A process according to claim 8, wherein the cells are harvested at about 35% to about 40% cell viability or after about 276 to about 300 hours.
